SEQUENCE LISTING (123 sequences)

SEQ ID NO: 1 - RNA, 22 nt, Artificial Sequence
  synthetic oligonucleotide
daaagaacucgaguugaugccg

SEQ ID NO: 2 - RNA, 21 nt, Artificial Sequence
  synthetic oligonucleotide
aagaacucgaguugaugccgg

SEQ ID NO: 3 - RNA, 21 nt, Artificial Sequence
  synthetic oligonucleotide
aaaaaggccugaaggaggagg

SEQ ID NO: 4 - RNA, 21 nt, Artificial Sequence
  synthetic oligonucleotide
aaaaggccugaaggaggagga

SEQ ID NO: 5 - RNA, 21 nt, Artificial Sequence
  synthetic oligonucleotide
aaaaagacggagugugcagag

SEQ ID NO: 6 - RNA, 21 nt, Artificial Sequence
  synthetic oligonucleotide
aaaagacggagugugcagaga

SEQ ID NO: 7 - RNA, 21 nt, Artificial Sequence
  synthetic oligonucleotide
aaagacggagugugcagagag

SEQ ID NO: 8 - RNA, 21 nt, Artificial Sequence
  synthetic oligonucleotide
aagacggagugugcagagaga

SEQ ID NO: 9 - RNA, 21 nt, Artificial Sequence
  synthetic polynucleotide
aagcuguggaguugcaggaua

SEQ ID NO: 10 - RNA, 21 nt, Artificial Sequence
  synthetic polynucleotide
aaacgcucacuucaggaaaua

SEQ ID NO: 11 - RNA, 21 nt, Artificial Sequence
  synthetic polynucleotide
aacgcucacuucaggaaauac

SEQ ID NO: 12 - RNA, 21 nt, Artificial Sequence
  synthetic polynucleotide
aaaugcuccaaguguggcaaa

SEQ ID NO: 13 - RNA, 21 nt, Artificial Sequence
  synthetic polynucleotide
aaccugcacagacauucggag

SEQ ID NO: 14 - RNA, 21 nt, Artificial Sequence
  synthetic polynucleotide
aacaagaaagaggaagcagac

SEQ ID NO: 15 - RNA, 21 nt, Artificial Sequence
  synthetic polynucleotide
aaaaagacggagugugcagag

SEQ ID NO: 16 - RNA, 21 nt, Artificial Sequence
  synthetic polynucleotide
aagaaagaggaagcagaccau

SEQ ID NO: 17 - RNA, 21 nt, Artificial Sequence
  synthetic polynucleotide
aaagaggaagcagaccauccu

SEQ ID NO: 18 - RNA, 21 nt, Artificial Sequence
  synthetic polynucleotide
aagaggaagcagaccauccug

SEQ ID NO: 19 - RNA, 21 nt, Artificial Sequence
  synthetic polynucleotide
aaaccacagccagagucaaag

SEQ ID NO: 20 - RNA, 21 nt, Artificial Sequence
  synthetic polynucleotide
aaagaggaaguggaugaaggc

SEQ ID NO: 21 - RNA, 21 nt, Artificial Sequence
  synthetic polynucleotide
aaaagacggagugugcagaga

SEQ ID NO: 22 - RNA, 21 nt, Artificial Sequence
  synthetic polynucleotide
aacacgauggauaagugagag

SEQ ID NO: 23 - RNA, 21 nt, Artificial Sequence
  synthetic polynucleotide
aagugagagagagucagguug

SEQ ID NO: 24 - RNA, 21 nt, Artificial Sequence
  synthetic polynucleotide
aaauagucuagaccagcuagu

SEQ ID NO: 25 - RNA, 21 nt, Artificial Sequence
  synthetic polynucleotide
aauagucuagaccagcuagug

SEQ ID NO: 26 - RNA, 21 nt, Artificial Sequence
  synthetic polynucleotide
aauuaugcuccuuggcaggua

SEQ ID NO: 27 - RNA, 21 nt, Artificial Sequence
  synthetic polynucleotide
aaggcaaauguguaccuguaa

SEQ ID NO: 28 - RNA, 21 nt, Artificial Sequence
  synthetic polynucleotide
aagcucgaggaagagcaggag

SEQ ID NO: 29 - RNA, 21 nt, Artificial Sequence
  synthetic polynucleotide
aagaaccaguuauuggcugaa

SEQ ID NO: 30 - RNA, 21 nt, Artificial Sequence
  synthetic polynucleotide
aaccaguuauuggcugaaaga

SEQ ID NO: 31 - RNA, 21 nt, Artificial Sequence
  synthetic polynucleotide
aaagaacaaaggagcagcucu

SEQ ID NO: 32 - RNA, 21 nt, Artificial Sequence
  synthetic polynucleotide
aaagacggagugugcagagag

SEQ ID NO: 33 - RNA, 21 nt, Artificial Sequence
  synthetic polynucleotide
aaugucaggagaugaaagaag

SEQ ID NO: 34 - RNA, 21 nt, Artificial Sequence
  synthetic polynucleotide
aagugacgaaauuguucucac

SEQ ID NO: 35 - RNA, 21 nt, Artificial Sequence
  synthetic polynucleotide
aaauguggaagaacaagagga

SEQ ID NO: 36 - RNA, 21 nt, Artificial Sequence
  synthetic polynucleotide
aauguggaagaacaagaggau

SEQ ID NO: 37 - RNA, 21 nt, Artificial Sequence
  synthetic polynucleotide
aagaacaagaggaucaaccua

SEQ ID NO: 38 - RNA, 21 nt, Artificial Sequence
  synthetic polynucleotide
aaaccuuccguacggucacuc

SEQ ID NO: 39 - RNA, 21 nt, Artificial Sequence
  synthetic polynucleotide
aaauguuccaugugcaaguau

SEQ ID NO: 40 - RNA, 21 nt, Artificial Sequence
  synthetic polynucleotide
aaguaugccaguguggaggca

SEQ ID NO: 41 - RNA, 21 nt, Artificial Sequence
  synthetic polynucleotide
aauugaagcgccauguccgau

SEQ ID NO: 42 - RNA, 21 nt, Artificial Sequence
  synthetic polynucleotide
aagacggagugugcagagaga

SEQ ID NO: 43 - RNA, 21 nt, Artificial Sequence
  synthetic polynucleotide
aaacgccacaugagaacgcac

SEQ ID NO: 44 - RNA, 21 nt, Artificial Sequence
  synthetic polynucleotide
aacgccacaugagaacgcacu

SEQ ID NO: 45 - RNA, 21 nt, Artificial Sequence
  synthetic polynucleotide
aacuugcaugcuuacagcgcu

SEQ ID NO: 46 - RNA, 21 nt, Artificial Sequence
  synthetic polynucleotide
aagcaggaacgucauaugacc

SEQ ID NO: 47 - RNA, 21 nt, Artificial Sequence
  synthetic polynucleotide
aaauguuuccgacagaagcaa

SEQ ID NO: 48 - RNA, 21 nt, Artificial Sequence
  synthetic polynucleotide
aagcuguggaguugcaggaua

SEQ ID NO: 49 - RNA, 21 nt, Artificial Sequence
  synthetic polynucleotide
aauguuuccgacagaagcaac

SEQ ID NO: 50 - RNA, 21 nt, Artificial Sequence
  synthetic polynucleotide
aagcaacuucuaaacgcucac

SEQ ID NO: 51 - RNA, 21 nt, Artificial Sequence
  synthetic polynucleotide
aaugcuccaaguguggcaaag

SEQ ID NO: 52 - RNA, 21 nt, Artificial Sequence
  synthetic polynucleotide
aagcaaagucggcugcuucag

SEQ ID NO: 53 - RNA, 21 nt, Artificial Sequence
  synthetic polynucleotide
aaagucggcugcuucaggaaa

SEQ ID NO: 54 - RNA, 21 nt, Artificial Sequence
  synthetic polynucleotide
aagaagaacaagaaagaggaa

SEQ ID NO: 55 - RNA, 21 nt, Artificial Sequence
  synthetic polynucleotide
aagaacaagaaagaggaagca

SEQ ID NO: 56 - RNA, 21 nt, Artificial Sequence
  synthetic polynucleotide
aagcagaccauccugaaggaa

SEQ ID NO: 57 - RNA, 21 nt, Artificial Sequence
  synthetic polynucleotide
aaccacagccagagucaaaga

SEQ ID NO: 58 - RNA, 21 nt, Artificial Sequence
  synthetic polynucleotide
aagaggaaguggaugaaggcg

SEQ ID NO: 59 - DNA, 21 bp, Artificial Sequence
  synthetic RNA/DNA polynucleotide; 19 ribonucleotides with two 3′ deoxyribonucleotides (thymidine)
ggaaauaccacgaugcaaatt

SEQ ID NO: 60 - DNA, 21 bp, Artificial Sequence
  synthetic RNA/DNA polynucleotide; 19 ribonucleotides with two 3′ deoxyribonucleotides (thymidine)
ggcaaguaaauugaagcgctt

SEQ ID NO: 61 - DNA, 21 bp, Artificial Sequence
  synthetic RNA/DNA polynucleotide; 19 ribonucleotides with two 3′ deoxyribonucleotides (thymidine)
ggaucaaccuacagcuggutt

SEQ ID NO: 62 - DNA, 21 bp, Artificial Sequence
  synthetic RNA/DNA polynucleotide; 19 ribonucleotides with two 3′ deoxyribonucleotides (thymidine)
uuugcaucgugguauuucctg

SEQ ID NO: 63 - DNA, 21 bp, Artificial Sequence
  synthetic RNA/DNA polynucleotide; 19 ribonucleotides with two 3′ deoxyribonucleotides (thymidine)
gcgcuucaauuuacuugcctc

SEQ ID NO: 64 - DNA, 21 bp, Artificial Sequence
  synthetic RNA/DNA polynucleotide; 19 ribonucleotides with two 3′ deoxyribonucleotides (thymidine)
accagcuguagguugaucctc

SEQ ID NO: 65 - DNA, 21 bp, Artificial Sequence
  synthetic polynucleotide
aaggaaccttccactgtgatg

SEQ ID NO: 66 - DNA, 21 bp, Artificial Sequence
  synthetic polynucleotide
aggaaccttccactgtgatgt

SEQ ID NO: 67 - DNA, 21 bp, Artificial Sequence
  synthetic polynucleotide
ggaaccttccactgtgatgtc

SEQ ID NO: 68 - DNA, 21 bp, Artificial Sequence
  synthetic polynucleotide
accttccactgtgatgtctgc

SEQ ID NO: 69 - DNA, 21 bp, Artificial Sequence
  synthetic polynucleotide
ccttccactgtgatgtctgca

SEQ ID NO: 70 - DNA, 21 bp, Artificial Sequence
  synthetic polynucleotide
ctgtgatgtctgcatgttcac

SEQ ID NO: 71 - DNA, 21 bp, Artificial Sequence
  synthetic polynucleotide
tgtctgcatgttcacctcttc

SEQ ID NO: 72 - DNA, 21 bp, Artificial Sequence
  synthetic polynucleotide
tgttcacctcttctagaatgt

SEQ ID NO: 73 - DNA, 21 bp, Artificial Sequence
  synthetic polynucleotide
cctcttctagaatgtcaagtt

SEQ ID NO: 74 - DNA, 21 bp, Artificial Sequence
  synthetic RNA/DNA polynucleotide; 19 ribonucleotides with two 3′ deoxyribonucleotides (thymidine)
caaaggagcagcucuuuuutt

SEQ ID NO: 75 - DNA, 21 bp, Artificial Sequence
  synthetic RNA/DNA polynucleotide; 19 ribonucleotides with two 3′ deoxyribonucleotides (thymidine)
aggagcagcucuuuuuugutt

SEQ ID NO: 76 - DNA, 21 bp, Artificial Sequence
  synthetic RNA/DNA polynucleotide; 19 ribonucleotides with two 3′ deoxyribonucleotides (thymidine)
acaaugucaggagaugaaatt

SEQ ID NO: 77 - DNA, 21 bp, Artificial Sequence
  synthetic RNA/DNA polynucleotide; 19 ribonucleotides with two 3′ deoxyribonucleotides (thymidine)
ugucaggagaugaaagaagtt

SEQ ID NO: 78 - DNA, 21 bp, Artificial Sequence
  synthetic RNA/DNA polynucleotide; 19 ribonucleotides with two 3′ deoxyribonucleotides (thymidine)
agaagugacgaaauuguuctt

SEQ ID NO: 79 - DNA, 21 bp, Artificial Sequence
  synthetic RNA/DNA polynucleotide; 19 ribonucleotides with two 3′ deoxyribonucleotides (thymidine)
gugacgaaauuguucucactt

SEQ ID NO: 80 - DNA, 21 bp, Artificial Sequence
  synthetic RNA/DNA polynucleotide; 19 ribonucleotides with two 3′ deoxyribonucleotides (thymidine)
auuguucucacaguuucaatt

SEQ ID NO: 81 - DNA, 21 bp, Artificial Sequence
  synthetic RNA/DNA polynucleotide; 19 ribonucleotides with two 3′ deoxyribonucleotides (thymidine)
auucaaauguggaagaacatt

SEQ ID NO: 82 - DNA, 21 bp, Artificial Sequence
  synthetic RNA/DNA polynucleotide; 19 ribonucleotides with two 3′ deoxyribonucleotides (thymidine)
auguggaagaacaagaggatt

SEQ ID NO: 83 - DNA, 21 bp, Artificial Sequence
  synthetic RNA/DNA polynucleotide; 19 ribonucleotides with two 3′ deoxyribonucleotides (thymidine)
aauguccccaaauaccagutt

SEQ ID NO: 84 - DNA, 21 bp, Artificial Sequence
  synthetic RNA/DNA polynucleotide; 19 ribonucleotides with two 3′ deoxyribonucleotides (thymidine)
aagcgaccuacgugugcautt

SEQ ID NO: 85 - DNA, 21 bp, Artificial Sequence
  synthetic RNA/DNA polynucleotide; 19 ribonucleotides with two 3′ deoxyribonucleotides (thymidine)
cuugcaugcuuacagcgcutt

SEQ ID NO: 86 - DNA, 21 bp, Artificial Sequence
  synthetic RNA/DNA polynucleotide; 19 ribonucleotides with two 3′ deoxyribonucleotides (thymidine)
aacucauaagaaugagaagtt

SEQ ID NO: 87 - DNA, 21 bp, Artificial Sequence
  synthetic RNA/DNA polynucleotide; 19 ribonucleotides with two 3′ deoxyribonucleotides (thymidine)
gaaugagaagagguucaagtt

SEQ ID NO: 88 - DNA, 21 bp, Artificial Sequence
  synthetic RNA/DNA polynucleotide; 19 ribonucleotides with two 3′ deoxyribonucleotides (thymidine)
gugcaaacacugcaguuautt

SEQ ID NO: 89 - DNA, 21 bp, Artificial Sequence
  synthetic RNA/DNA polynucleotide; 19 ribonucleotides with two 3′ deoxyribonucleotides (thymidine)
gcaggaacgucauaugacctt

SEQ ID NO: 90 - DNA, 21 bp, Artificial Sequence
  synthetic RNA/DNA polynucleotide; 19 ribonucleotides with two 3′ deoxyribonucleotides (thymidine)
auguuuccgacagaagcaatt

SEQ ID NO: 91 - DNA, 21 bp, Artificial Sequence
  synthetic RNA/DNA polynucleotide; 19 ribonucleotides with two 3′ deoxyribonucleotides (thymidine)
acgcucacuucaggaaauatt

SEQ ID NO: 92 - DNA, 21 bp, Artificial Sequence
  synthetic RNA/DNA polynucleotide; 19 ribonucleotides with two 3′ deoxyribonucleotides (thymidine)
auaccacgaugcaaauuuctt

SEQ ID NO: 93 - DNA, 21 bp, Artificial Sequence
  synthetic RNA/DNA polynucleotide; 19 ribonucleotides with two 3′ deoxyribonucleotides (thymidine)
auuucaucccgacuguuuatt

SEQ ID NO: 94 - RNA, 19 nt, Artificial Sequence
  synthetic polynucleotide
uuucgucacuucuuucauc

SEQ ID NO: 95 - RNA, 19 nt, Artificial Sequence
  synthetic polynucleotide
ucucucucacuuauccauc

SEQ ID NO: 96 - RNA, 19 nt, Artificial Sequence
  synthetic polynucleotide
ucuuucuuguucuucuucc

SEQ ID NO: 97 - RNA, 19 nt, Artificial Sequence
  synthetic polynucleotide
ugacucucucucacuuauc

SEQ ID NO: 98 - RNA, 19 nt, Artificial Sequence
  synthetic polynucleotide
uuguucuucuucccuuucc

SEQ ID NO: 99 - RNA, 19 nt, Artificial Sequence
  synthetic polynucleotide
uuucuggugcugaaugagg

SEQ ID NO: 100 - RNA, 19 nt, Artificial Sequence
  synthetic polynucleotide
uuucaucuccugacauugu

SEQ ID NO: 101 - RNA, 19 nt, Artificial Sequence
  synthetic polynucleotide
ucaugaguauguuuauagc

SEQ ID NO: 102 - RNA, 19 nt, Artificial Sequence
  synthetic polynucleotide
uucagcuuguagguaucuc

SEQ ID NO: 103 - RNA, 19 nt, Artificial Sequence
  synthetic polynucleotide
uuuagaaguugcuucuguc

SEQ ID NO: 104 - RNA, 19 nt, Artificial Sequence
  synthetic polynucleotide
ucuagacacuaccucaaac

SEQ ID NO: 105 - RNA, 19 nt, Artificial Sequence
  synthetic polynucleotide
uuugcaucgugguauuucc

SEQ ID NO: 106 - RNA, 19 nt, Artificial Sequence
  synthetic polynucleotide
uauuuggggacauuuucgc

SEQ ID NO: 107 - RNA, 19 nt, Artificial Sequence
  synthetic polynucleotide
auuguuucaaagaaaaugc

SEQ ID NO: 108 - RNA, 19 nt, Artificial Sequence
  synthetic polynucleotide
auaugcacacguaggucgc

SEQ ID NO: 109 - RNA, 19 nt, Artificial Sequence
  synthetic polynucleotide
auucagucacaaugauggc

SEQ ID NO: 110 - RNA, 19 nt, Artificial Sequence
  synthetic polynucleotide
uaaaaacuucuaacuugcu

SEQ ID NO: 111 - RNA, 19 nt, Artificial Sequence
  synthetic polynucleotide
aagacagcagaacaguagc

SEQ ID NO: 112 - RNA, 19 nt, Artificial Sequence
  synthetic polynucleotide
uuauugcaagaaaggcagg

SEQ ID NO: 113 - RNA, 19 nt, Artificial Sequence
  synthetic polynucleotide
uuaauccagcgggaaaagc

SEQ ID NO: 114 - RNA, 19 nt, Artificial Sequence
  synthetic polynucleotide
uugaggagcauuucacacc

SEQ ID NO: 115 - RNA, 19 nt, Artificial Sequence
  synthetic polynucleotide
uuaugaguuuucuggugcu

SEQ ID NO: 116 - RNA, 19 nt, Artificial Sequence
  synthetic polynucleotide
uugaaccucuucucauucu

SEQ ID NO: 117 - RNA, 19 nt, Artificial Sequence
  synthetic polynucleotide
auaugacguuccugcuugc

SEQ ID NO: 118 - RNA, 19 nt, Artificial Sequence
  synthetic polynucleotide
aacaauucuagacacuacc

SEQ ID NO: 119 - RNA, 19 nt, Artificial Sequence
  synthetic polynucleotide
uucuucccuuuccugaagc

SEQ ID NO: 120 - RNA, 19 nt, Artificial Sequence
  synthetic polynucleotide
aaccugacucucucucacu

SEQ ID NO: 121 - RNA, 19 nt, Artificial Sequence
  synthetic polynucleotide
augaguauguuuauagcgc

SEQ ID NO: 122 - RNA, 19 nt, Artificial Sequence
  synthetic polynucleotide
auucuuaugaguuuucugg

SEQ ID NO: 123 - RNA, 19 nt, Artificial Sequence
  synthetic polynucleotide
uaacugcaguguuugcacu

(19) United States
(12) Patent Application Publication
(10) Pub. No.: US 2009/0169613 A1
(43) Pub. Date: Jul. 2, 2009

(54) TARGETING OF TUMOR STEM CELLS THROUGH SELECTIVE SILENCING OF BORIS EXPRESSION

(76) Inventors: Boris N. Reznik, Aventura, FL (US); Thomas Ichim, San Diego, CA (US); Christopher Dougherty, Lake Worth, FL (US)

Correspondence Address: The Law Office of Jane K. Babin;Professional Corporation, c/o Intellevate, P.O. Box 52050, Minneapolis, MN, 55402, US

(21) Appl. No.: 12/268,399

(22) Filed: Nov. 10, 2008

Related U.S. Application Data
(60) Provisional application No. 60/986,623, filed on Nov. 9, 2007.

(51) Int. Cl. A61K 9/127; A61K 39/395; A61K 38/16; A61K 9/14; A61P 35/00

(52) U.S. Cl. 424/450; 424/174.1; 514/12; 424/489; 977/734; 977/773

(57) ABSTRACT

The present invention provides compositions useful for the treatment of cancer that inhibit tumor stem cells through suppression of an activity or the expression of BORIS. The compositions target tumor stem cells through molecules that are specific to tumor stem cells. Specifically, the invention provides immunoliposomes specific to tumor stem cells that include nucleic acid compositions capable of eliciting the process of RNA interference of BORIS expression. Also provided are immunoliposomes specific to tumor stem cells that include anti-BORIS ribozymes, antisense oligonucleotides, decoy oligonucleotides or small molecule inhibitors. Methods of manufacturing, delivering, and use of such compositions in the treatment of cancer are also provided.

### RELATED APPLICATIONS

The present application claims the benefit of priority to U.S. Provisional Application No. 60/986,623 filed Nov. 9, 2007, the entire contents of which are incorporated herein by reference.

### FIELD OF THE INVENTION

The current invention relates to the field of cancer therapeutics, and particularly to therapeutic targeting of tumor stem cells. Furthermore, the invention relates to cancer therapeutics such as nucleic acids, such as siRNAs to cancer stem cells by the used of immunoliposomes or molecules containing a cancer stem cell targeting moiety.

### BACKGROUND

Selective targeting of therapeutic reagents to tumor tissues has previously been attempted using immunological, metabolic, and molecular biology approaches, but with limited success. Among the major reasons for failure of such tumor-targeting therapies are the failure to identify tumor targets that are selective for the tumor versus non-tumor cells and essential for maintenance of tumor phenotype, the inability to inactivate targets, and the failure to kill cells expressing the target. Nevertheless, there remains an interest in developing selectively targeted therapies for cancer.

### The Brother of the Regulator of Imprinted Sites (BORIS)

BORIS is an 11-zinc finger protein that is specifically expressed in neoplastically-transformed tissues, including tumor cell lines and primary patient samples, but is not expressed in non-transformed tissues with the exception of testis. The BORIS gene encodes a germ line, testis- and cancer-specific, paralog of the CTCF (CCCTC-binding factor; GenBank Accession No.: NM_{—}006565), and is an epigentically-acting transcription factor that represses the tumor inhibitor functions of CTCF. Thus, BORIS is also referred to as CTCFL for CTCF-like. BORIS contains a central DNA-binding domain that is nearly identical to CTCF, but differs in N and C termini amino acid sequence, thereby suggesting that BORIS could play a role of interfering with CTCF-driven regulatory pathways if it is abnormally expressed in somatic cells (Klenova et al., Semin. Cancer Biol. (2002) 12:399-414; Loukinov et al., Proc. Natl. Acad. Sci. USA (2002) 99:6806-11).

Abnormal activation of BORIS has been observed in all human primary tumors and cancer cell lines tested, including breast, lung, skin, bone, brain, colon, prostate, pancreas, mast cell, ovarian and uterine cancers, with increased expression associated with advanced stage of disease (see e.g., Ulaner et al., Hum. Mol. Genet. (2003)12:535-49; Vatolin et al., Cancer Res (2005) 65:7751-62; Hong et al., Cancer Res (2005) 65:7763-74; and Loukinov et al, J. Cell. Biochem. (2006) 98:1037-43; D'Arcy et al, Br. J. Cancer (2008) 98:571-9). BORIS induces de-repression of many genes associated with malignancy (Vatolin et al., Cancer Res (2005) 65:7751-62; Hong et al., Cancer Res (2005) 65:7763-74), and ectopic expression of BORIS in normal cells has been reported to result in classic features of cell-transformation (see Ghochikyan et al., J. Immunol. (2007) 178: 566-73).

BORIS reportedly competes with CTCF for shared DNA target sites and can tether epigenetic modifications to and around such sites, resulting in modulation of gene expression (see Vatolin et al., Cancer Res (2005) 65:7751-62; Hong et al., Cancer Res (2005) 65:7763-74; Ghochikyan et al., J. Immunol. (2007) 178: 566-73). BORIS can also bind methylated DNA target sites, while there is evidence that CTCF cannot (see Nguyen et al., Cancer Res (2008) 68:5546-51). Therefore, BORIS can be classified as a unique cancer-testis gene with cell-transforming activity that is most likely mediated by competition with somatic tumor suppressor CTCF through epigenetic modifications (Vatolin et al., Cancer Res (2005) 65:7751-62). Interestingly, expression of BORIS has been correlated to the aggressiveness of tumor phenotype in uterine cancers.

Previous studies have demonstrated the potential of BORIS as a target for anti-cancer therapeutics. Protein-based, but not DNA-based, BORIS vaccine induced a significant level of antibody production in immunized animals, leading to breast cancer regression. Interestingly, potent anticancer CD8⁺-cytotoxic lymphocytes were generated after immunization with a DNA-based, but not protein-based, BORIS vaccine. (Ghochikyan et al., J. Immunol. (2007) 178: 566-73). However, the applicability of immunological approaches to cancer treatment is subject to limitations, including a) tumor suppression of the host immune system through active production of soluble and membrane bound factors; b) ability of tumor cells to lose expression of antigen processing machinery; and c) possibility of a deficit in the immunological repertoire of cancer patients caused by down regulation of TCR zeta chain expression.

BORIS is a particularly appealing target for cancer therapy for several reasons. First, the widespread distribution of BORIS in different types of cancer cells coupled with the general concept that while non-malignant cells do not require activated oncogenes for survival, the suppression of an activated oncogene in a cancer cell often leads to apoptosis, suggests that therapies targeting BORIS may be effective for selective killing of a large number of cancer cell types. Thus, a single approach to cancer therapy may be applicable to many forms of cancer. Furthermore, BORIS is limited to testes and cancer cell types, and is not found in the vast majority of normal cell types. Therapies directed at BORIS are expected to have fewer side effects than others that target molecules or mechanisms present in normal cells, particularly in women where BORIS is not found in normal tissues.

The physiological function of BORIS is reportedly related to erasure of methylation patterns during the process of spermatogenesis and hence the only expression of this gene in normal tissues is in the testis. BORIS is reportedly an epigenetic-acting oncogene that is thought to induce derepression of other oncogenes by inhibiting activity of the tumor suppressor gene, the CCCTC-binding factor (CTCF). CTCF was originally identified by its ability to suppress expression of the c-myc oncogene. Specifically, CTCF protein was reported to selectively bind CCCTC repeats in DNA upstream of the c-myc transcription start site. Deletion of CTCF binding regions was associated with upregulation of c-myc transcription. CTCF has also been reported to repress transcription of additional oncogenes including p27, p21, p53, p19 (ARF) and telomerase. The importance of CTCF as a tumor suppressor gene has been demonstrated by studies showing that mutation of CTCF results in oncogenesis and the finding that tumors express mutated CTCF. It has been speculated that BORIS selectively inactivates CTCF activity, thereby derepressing transcription of various oncogenes which ultimately results in the process of oncogenesis (4, 12).

### RNA Interference

RNA interference (RNAi) is a process by which a double-stranded RNA (dsRNA) selectively inactivates homologous mRNA transcripts by triggering specific degradation of homologous RNAs in the cell. RNAi is more potent than anti-sense technology, giving effective knockdown of gene expression with as little as 1-3 molecules of duplex RNA per cell. Furthermore, inhibition of gene expression can migrate from cell to cell and may even be passed from one generation of cells to another.

Traditionally, RNAi has required long pieces (200-800 base pairs) of dsRNA to be effective. This is impractical for therapeutic uses due to the sensitivity of long RNA molecules to cleavage by RNases found in the plasma and intracellularly. In addition, long pieces of dsRNA have been reported to induce panic responses in eukaryotic cells, which include nonspecific inhibition of gene transcription, but also production of interferon-α.

When long dsRNA duplexes enter the cytoplasm, an RNase III type enzymatic activity cleaves the duplex into smaller, 21-23 base-pairs molecules, termed small interfering RNA (siRNA). Short RNA duplexes are active in silencing gene expression but do not trigger nonspecific panic responses when less than 30 nucleotides in length. Moreover, siRNAs can be administered directly to a cell or organism to silence gene expression, thereby obviating the need to use long dsRNA or less effective single-strand anti-sense RNA, ribozymes, or the like.

siRNA has been found effective for inhibiting expression of a variety of genes in mammalian cells in vitro and in vivo. siRNA technology provides an appealing approach for selectively inhibiting gene expression in clinical and therapeutic settings due to many advantages over conventional gene and antibody blocking approaches, including: (1) potent inhibitory efficacy; (2) specificity—even a single nucleotide mismatch can be distinguished; (3) inhibitory effects that can be passed to daughter cells for multiple generations; (4) high in vitro transfection efficiency; (5) practicality for in vivo use due to short sequence length, low effective concentrations and lack of neutralizing antibody production; (6) availability of tissue- and cell-specific targeting (e.g. via inducible or promoter-specific vectors, ligand-directed liposomes or antibody-conjugated liposomes); and (7) possibility of simultaneously silencing multiple genes or multiple exons in a single gene.

### SUMMARY OF THE INVENTION

The present invention provides compositions for the treatment of cancer that include: a) at least one molecule specific to a tumor stem cell; b) a carrier bound to the at least one molecule of a); and c) at least one molecule capable of suppressing transcription, translation or a function of i) the Brother of the Regulator of Imprinted Sites (BORIS) molecule, or ii) an isoform of BORIS. The molecule specific to a tumor stem cell can be, without limitation, an antibody, an aptamer, a fusion protein, or a small organic compound. Antibodies contemplated for use in the compositions of the invention include those recognizing and/or directed to CD133, decay accelerating factor, CD117, prostate stem cell antigen, CD44, CD29, alpha6-integrin, CD200, stem cell antigen, or multiple drug resistance protein.

In certain embodiments, the carrier portion of the compositions of the invention is a liposome, a fullerene molecule, a cationic lipid particle, a biodegradable nanoparticle, or an aerosolized particle.

Molecules capable of suppressing transcription, translation or a function BORIS include antisense oligonucleotides, short interfering RNAs, ribozymes, molecules that prevent BORIS from binding to DNA, molecules that prevent binding of BORIS to co-factors, and molecules that prevent recruitment of cofactors needed for BORIS transcription. These molecules can, for example, include modified or unmodified nucleic acids, peptides and/or small organic compounds.

In certain embodiments of the invention, the compositions include polyethelyne glycol-based immunoliposomes containing at least one anti-CD133 antibody loaded with siRNA targeting the BORIS gene. To facilitate conjugation to the immunoliposome, the antibody, can be for example, thiolated.

In certain embodiments, one strand of the siRNA has a nucleotide sequence selected from SEQ ID NOs:1-61 or from SEQ ID NOs:62-123. The siRNA molecule can be synthesized from a polynucleotide that encodes the siRNA molecule or a precursor of the siRNA.

Certain compositions of the invention contain an immunoliposome comprising a thiolated antibody that binds CD133 coupled to the distal reactive maleimide terminus of a poly (ethylene glycol)-phospholipid conjugate so that the antibody is partially incorporated into liposomal bilayer, and a nucleic acid sequence capable of selectively inhibiting expression or an activity of BORIS, encapsulated or inserted therein. For example, the nucleic acid can have the nucleotide sequence of SEQ ID NOs:59 or 60. In certain embodiments, the immunoliposomes of the invention have a particle size of about 50 to about 400 nanometers. Also contemplated by the invention are admixtures of compositions of the invention with cytotoxic agents.

In yet further embodiments the compositions can also contain at least one molecule specific to a tumor cell, such as a tumor-specific antibody incorporated into the carrier (e.g., immunoliposome).

Also provided by the invention are methods of treating cancer including administering the composition of the invention to a subject. The compositions can be administered alone or in combination with administration of chemotherapeutic agents, immunotherapeutic agents, hormonal therapeutic agents, radiation therapy, surgery, and/or embolization therapy.

### DETAILED DESCRIPTION

### Definitions

It is to be understood that both the foregoing general description and the following detailed description are exemplary and explanatory only and are not restrictive of the invention, as claimed. It must be noted that, as used herein and in the appended claims, the singular forms include plural referents; the use of “or” means “and/or” unless stated otherwise. Thus, for example, reference to “a subject polypeptide” includes a plurality of such polypeptides and reference to “the agent” includes reference to one or more agents and equivalents thereof known to those skilled in the art, and so forth. Moreover, it must be understood that the invention is not limited to the particular embodiments described, as such may, of course, vary. Further, the terminology used to describe particular embodiments is not intended to be limiting, since the scope of the present invention will be limited only by its claims.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

Unless otherwise defined, all technical and scientific terms used herein have the same meaning as commonly understood by one of ordinary skill in the art to which this invention belongs. Suitable methods and materials are described below, however methods and materials similar or equivalent to those described herein can be used in the practice of the present invention. Thus, the materials, methods, and examples are illustrative only and not intended to be limiting. All publications, patent applications, patents, and other references mentioned herein are incorporated by reference in their entirety. In case of conflict, the present specification, including definitions, will control.

Standard techniques may be used for recombinant DNA, oligonucleotide synthesis, tissue culture and transfection (e.g., electroporation, lipofection, etc.). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)); Current Protocols in Molecular Biology (eds. Ausubel, et al., Greene Publ. Assoc., Wiley-Interscience, NY); Harlow and Lane, Antibodies, A Laboratory Manual, Cold Spring Harbor Laboratory, Cold Spring Harbor, N.Y. (1988)) the entire contents of which are incorporated herein by reference for any purpose. Unless specific definitions are provided, the nomenclatures utilized in connection with, and the laboratory procedures and techniques of, analytical chemistry, synthetic organic chemistry, and medicinal and pharmaceutical chemistry described herein are those well known and commonly used in the art. Standard techniques may be used for chemical syntheses, chemical analyses, pharmaceutical preparation, formulation, and delivery, and treatment of patients

### DEFINITIONS

“About” as used herein means that a number referred to as “about” comprises the recited number plus or minus 1-10% of that recited number. For example, “about” 100 nucleotides can mean 100 nucleotides, 99-101 nucleotides, or up to as broad a range as 90-100 nucleotides. Whenever it appears herein, a numerical range such as “1 to 100” refers to each integer in the given range; e.g., “1 to 100 nucleotides” means that the nucleic acid can contain only 1 nucleotide, 2 nucleotides, 3 nucleotides, etc., up to and including 100 nucleotides.

“BORIS” or “the Brother of the Regulator of Imprinted Sites” protein, as used herein, refers to an epigenetically-acting zinc finger polypeptide present in mammalian testes and cancer cells, with an amino acid sequence that has greater than about 80% amino acid sequence identity, typically greater than 85% identity, often greater than 90% identity, and preferably 91%, 92%, 93%, 94%, 95%, 96%, 97%, 98% or 99% or greater amino acid sequence identity, to the BORIS amino acid sequence detailed in GenBank Accession No. AAM28645 (posted May 16, 2002). Implicitly encompassed by this definition are splice variants, variants containing conservative amino acid substitutions, and polymorphic variants capable of transforming a mammalian cell. The skilled artisan will be aware of methods for determining whether a polymorphic variant of BORIS is capable of transforming a mammalian cell, such as by transfection of a nucleic acid encoding the variant into a cell and e.g. observing colony formation. Typically, cancer cells that express BORIS have the amino acid sequence of GenBank Accession No. AAM28645, a splice variant thereof, a variant containing one or more conservative amino acid substitutions, or a polymorphic variant thereof that is capable of transforming a mammalian cell.

Identity is determined over a region of at least 20, 50, 100, 200, 500, or more contiguous amino acids. The terms “identical” or percent “identity,” as used herein in the context of two or more nucleic acids or amino acid sequences, refer to two or more sequences or subsequences that are the same or have a specified percentage of amino acid residues or nucleotides that are the same when compared and aligned for maximum correspondence over a comparison window (i.e. region). The definition includes sequences that have deletions, insertions and substitutions and may also be applied to the complement of a sequence (e.g. “100% complementary” polynucleotides). Preferably, identity is measured over the length of the polynucleotide or polypeptide, but is typically measured over a region that is at least about 20 amino acids or nucleotides in length, and often over a region that is at least 50-100 amino acids or nucleotides in length.

To calculate percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value, which is usually rounded to the nearest integer. It will be appreciated that the length of the aligned region can be a portion of one or both sequences up to the full-length of the shortest sequence. It will be appreciated that a single sequence can align differently with other sequences and hence, can have different percent sequence identity values over each aligned region.

The alignment of two or more sequences to determine percent sequence identity can be performed manually, by visual alignment, or can use computer programs that are well known in the art. For example, the algorithm described by Altschul et al. (1997, Nucleic Acids Res., 25:3389 402) can be used. This algorithm is incorporated into BLAST (basic local alignment search tool) programs, available at ncbi.nlm.nih.gov on the World Wide Web. BLAST searches can be performed to determine percent sequence identity between a nucleic acid molecule or polypeptide of the invention and any other sequence or portion thereof.

“BORIS gene” or “BORIS polynucleotide” refer to a polynucleotide sequence encoding a BORIS polypeptide, which is transcribed into an mRNA with at least about 80% nucleotide sequence identity, typically greater than 85% identity, often greater than 90% identity, and preferably 91%, 92%, 93%, 94%, 95%, 96%, 97%, 98% or 99% or greater nucleotide sequence identity to the BORIS cDNA sequence of GenBank Accession No. AF336042 (posted May 16, 2002).

BORIS nucleic acid sequences also implicitly encompass “splice variants.” Similarly, BORIS polypeptides implicitly encompass any protein encoded by a splice variant of a BORIS nucleic acid. “Splice variant,” as used herein, refers to the products of alternative splicing of a gene. After transcription, an initial nucleic acid transcript may be spliced such that alternate nucleic acids are produced from the same template. Mechanisms for the production of splice variants include alternate splicing of exons. Alternate polypeptides derived from the same nucleic acid by read-through transcription are also encompassed by this definition. Any products of a splicing reaction, including recombinant forms of the splice products, are included in this definition.

“CTCF” as used herein refers to CCCTC-binding factor, a paralog of BORIS that is expressed in normal mammalian cells, and which typically has about 66% amino acid sequence identity to BORIS. “CTCF gene” refers to a polynucleotide sequence encoding a CTCF polypeptide, which is transcribed into an mRNA have a nucleotide sequence that has at least at least about 80% nucleotide sequence identity, typically greater than 85% identity, often greater than 90% identity, and preferably 91%, 92%, 93%, 94%, 95%, 96%, 97%, 98% or 99% or greater nucleotide sequence identity to the CTCF cDNA sequence of GenBank Accession No.: NM_{—}006565 (posted Jul. 20, 2008) or.

The terms “polynucleotide,” “nucleic acid,” and “nucleic acid molecule,” are used interchangeably herein to refer to polymeric forms of nucleotides of any length. The polynucleotides can contain deoxyribonucleotides, ribonucleotides, and/or their analogs. Polynucleotides can have any three-dimensional structure, and can perform any function, known or unknown. The term polynucleotide includes single-stranded, double-stranded, and triple helical molecules, and encompasses nucleic acids containing nucleotide analogs or modified backbone residues or linkages, which can be synthetic, naturally occurring, or non-naturally occurring, and which have similar binding properties as the reference nucleic acid. In particular, interfering RNAs (e.g., siRNA, shRNA) of the invention, can contain modifications or may incorporate analogs provided these do not interfere with the ability of the interfering RNA to inactivate homologous mRNA. Examples include replacement of one or more phosphodiester bonds with phosphorothioate linkages; modifications at the 2′-position of the pentose sugar in RNA, such as incorporation of 2′-O-methyl ribonucleotides, 2′-H ribonucleotides, 2′-deoxy-2′-fluoro ribonucleotides (e.g. 2′-deoxy-2′-fluorouridine), or 2′-deoxy ribonucleotides; incorporation of universal base nucleotides, 5-C-methyl nucleotides, inverted deoxyabasic residues, or locked nucleic acid (LNA), which contains a methylene linkage between the 2′ and the 4′ position of the ribose.

Exemplary embodiments of polynucleotides include, without limitation, genes, gene fragments, exons, introns, mRNA, tRNA, rRNA, interfering RNA, siRNA, shRNA, miRNA, anti-sense RNA, ribozymes, cDNA, recombinant polynucleotides, branched polynucleotides, plasmids, vectors, isolated DNA of any sequence, isolated RNA of any sequence, nucleic acid probes and primers.

“Oligonucleotide” refers generally to polynucleotides that are between 5 and about 100 nucleotides of single- or double-stranded DNA. For the purposes of this disclosure, the lower limit of the size of an oligonucleotide is two, and there is no upper limit to the length of an oligonucleotide. Oligonucleotides are also known as “oligomers” or “oligos” and can be prepared by any method known in the art including isolation from naturally-occurring polynucleotides, enzymatic synthesis and chemical synthesis.

The terms “polypeptide,” “peptide” and “protein” are used interchangeably herein to refer to a polymer of amino acid residues of any length. Polypeptides can have any three-dimensional structure, and can perform any function, known or unknown. The terms apply to amino acid polymers in which one or more amino acid residue is an artificial chemical mimetic of a corresponding naturally occurring amino acid, as well as to naturally occurring amino acid polymers and non-naturally occurring amino acid polymers.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The terms “conservatively modified variants” or “conservative variants” applies to both amino acid and nucleic acid sequences. With respect to particular nucleic acid sequences, conservatively modified variants refers to those nucleic acids which encode identical or substantially identical amino acid sequences; or for nucleic acids that do not encode an amino acid sequence, to nucleic acids that are substantially identical. As used herein, “substantially identical” means that two amino acid or polynucleotide sequences differ at no more than 10% of the amino acid or nucleotide positions, typically at no more than 5%, often at more than 2%, and most frequently at no more than 1% of the of the amino acid or nucleotide positions.

Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the alternate alanine codons without altering the encoded polypeptide. Such nucleic acid variations are “silent variations,” which are one type of conservatively modified variants. Nucleic acid sequences encoding polypeptides described herein also encompass every possible silent variation of the nucleic acid. The skilled artisan will recognize that each amino acid codon in a nucleic acid (except AUG, which is ordinarily the only codon for methionine, and TGG, which is ordinarily the only codon for tryptophan) can be varied at one or more positions to code for the same amino acid. Accordingly, each silent variation of a nucleic acid that encodes a polypeptide is implicit in each described sequence with respect to the expression product.

“Complementarity” refers to the ability of a nucleic acid to form hydrogen bond(s) with another polynucleotide sequence by either traditional Watson-Crick or other non-traditional types of base pairing. In reference to the nucleic molecules of the present invention, the binding free energy for a nucleic acid molecule with its target or complementary sequence is sufficient to allow the relevant function of the nucleic acid to proceed, e.g., enzymatic nucleic acid cleavage, RNA interference, antisense or triple helix inhibition. Determination of binding free energies for nucleic acid molecules is well known in the art. “Percent complementarity” refers to the percentage of contiguous residues in a nucleic acid molecule that can form hydrogen bonds (e.g., Watson-Crick base pairing) with another nucleic acid molecule. “Perfectly complementary” or “100% complementarity” means that all the contiguous nucleotides of a nucleic acid molecule will hydrogen bond with the same number of contiguous residues in a second nucleic acid molecule. “Substantial complementarity” and “substantially complementary” as used herein indicate that two nucleic acids are at least 90% complementary, typically at least 95% complementary, often at least 98% complementary, and most frequently at least 99% complementary over a region of more than about 15 nucleotides and more often more than about 19 nucleotides.

“Homology” is an indication that two nucleotide sequences represent the same gene or a gene product thereof, and typically means that that the nucleotide sequence of two or more nucleic acid molecules are partially, substantially or completely identical. When from the same organism, homologous polynucleotides are representative of the same gene having the same chromosomal location, even though there may be individual differences between the polynucleotide sequences (such as polymorphic variants, alleles and the like). In certain embodiments, a homolog can be found in a non-native position in the genome, e.g. as the result of translocation. Isolated and/or synthetic polynucleotides of the invention may be selected or designed to be homologous to an mRNA product of a gene. Preferably, homologous interfering RNAs of the invention are substantially identical to a target genomic DNA or mRNA sequence, but sufficiently different from other sequences in the genome so that they do not elicit an RNA interference effect with off-target polynucleotides.

Regarding amino acid sequences, one of skill in the art will recognize that individual substitutions, deletions or insertions to a nucleic acid, peptide, polypeptide, or protein sequence which alters, inserts or deletes a single amino acid or a small percentage of amino acids in the encoded sequence is a “conservatively modified variant” where the alteration results in the substitution of an amino acid with a chemically similar amino acid. Conservative substitution tables detailing functionally similar amino acids are well known in the art. Such conservatively modified variants are in addition to and do not exclude functionally equivalent polymorphic variants, homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

The term “RNA interference” or “RNAi” is broadly defined herein to include all posttranscriptional mechanisms of double-strand RNA mediated inhibition of gene expression. RNAi includes mechanisms that utilize siRNA and shRNA, as well as longer forms of duplex RNA. RNA interference is used to inhibit the function of an endogenous gene product, and thus mimic the effect of a loss-of-function mutation.

A “small interfering RNA” or “siRNA” is a double-stranded polynucleotide (e.g. RNA) molecule that mediates inhibition of the expression of a gene with which it shares homology when present in the same cell as the gene (i.e., target gene). siRNAs of the invention inhibit gene expression by directing cleavage of the target region of a homologous polynucleotide.

The region of the gene or other nucleotide sequence over which there is homology is known as the “target region.” siRNA thus refers to the double-stranded polynucleotides formed by short, complementary strands of polynucleotide. The complementary regions of nucleic acid sequence that hybridize to form duplex polynucleotide molecules typically have substantial or complete complementarity to each other and are homologous to a target region of a gene (e.g., the BORIS gene). In one embodiment, an siRNA refers to a nucleic acid that has substantial or complete identity to a target gene and forms a double-stranded oligonucleotide.

Typically, siRNAs of the invention are at least about 15-30 nucleotides in length, (e.g., each complementary polynucleotide of the double-stranded siRNA is 15-30 nucleotides in length, and the double-stranded siRNA is about 15-30 base pairs in length), typically about 19-24 nucleotides in length, most frequently about 21-22 nucleotides in length.

Endogenous siRNAs are produced from cleavage of longer double-strand RNA precursors by an RNaseIII endonuclease and have a characteristic 2 nucleotide 3′ overhang that allows them to be recognized by RNAi machinery, ultimately leading to homology-dependent cleavage of the target mRNA region. Cleavage is reportedly effected between bases 10 and 11 relative to the 5′ end of the antisense siRNA strand. Rules that govern selectivity of siRNA utilization by endogenous RNAi machinery are based upon differential thermodynamic stabilities of the ends of the siRNAs, with less thermodynamically stable ends favored. Such information can be valuable in selecting siRNA sequences from a target mRNA, which are then assessed for RNA interference activity according to the methods of the invention.

Partial complementarity between an siRNA and target mRNA may in certain cases repress translation or destabilize the transcripts if binding of the siRNA mimics microRNA (miRNA) interactions with their target sites. MicroRNAs are endogenous substrates for the RNAi machinery. Micro RNAs are initially expressed as long primary transcripts (pri-miRNAs), which are processed within the nucleus into 60-70 bp hairpins. The loop is removed by further processing in the cytoplasm by an RNase III activity. Mature miRNAs share only partial complementarity with sequences in the 3′UTR of target mRNAs. The primary mechanism of action of miRNAs is translational inhibition, although this can be accompanied by message degradation.

“Expression” or “gene expression” as used herein refers to the conversion of the information from a gene into a gene product. A gene product can be the direct transcriptional product of a gene (e.g., mRNA, tRNA, rRNA, antisense RNA, ribozyme, structural RNA, or any other type of RNA) or a protein produced by translation. According to the methods of the present invention, BORIS gene expression is typically measured by determining the amount BORIS polypeptide in the cell, such as by enzyme linked immunosorbent assay (ELISA), Western blotting, radioimmunoassay (RIA), immunofluorescence, fluorescence activated cell analysis (FACS) or other methods that utilize anti-BORIS antibodies. BORIS gene expression may also be detected using biochemical techniques for analyzing RNA such as Northern blotting, nuclease protection assays, reverse transcription, microarray hybridization, and the like, which are well known in the art. In other aspects of the invention, BORIS expression is determined by measuring an activity of BORIS, such as BORIS methylation activity, DNA binding activity, or cell transformation activity. In certain embodiments of the invention, the downstream effects of reduced BORIS gene expression may be measured as an indication of the inhibition of BORIS expression. Such downstream effects include reduced cell viability, cell death, increased apoptosis or the increased activity of apoptosis-related markers.

The terms “silencing,” “inhibition,” and “knockdown” of gene expression are used interchangeably herein to refer to a reduction in the amount of a BORIS gene product (i.e. BORIS polypeptide or BORIS mRNA) in a cell as a result of RNA interference. Inhibition indicates that expression of BORIS is reduced by 1-100% (e.g., 10%, 20%, 30%, 40%, 50%, 60%, 70%, 80%, 90%, 95%, or 99% reduced) compared to expression of BORIS in the absence of RNA interference.

As used herein, “sense” strand of an oligo- or polynucleotide refers to a molecule having a nucleotide sequence that is homologous to target mRNA strand, which target mRNA strand codes for a protein. In some embodiments, a sense strand is 100% identical to a sequence of the target mRNA. In other embodiments, a sense strand may be about 90%, about 95%, or about 99% identical to a target mRNA. An “antisense” strand refers to the complement of a sense strand or a target mRNA. In some embodiments, sense and antisense strands are 100% complementary to each. In other embodiments, the duplex polynucleotide, such as an siRNA, may contain one or more mismatched base pairs or terminal overhangs.

“Antibody” or “antibodies”, as used herein, include naturally occurring species such as polyclonal and monoclonal antibodies as well as any antigen-binding portion, fragment or subunit of a naturally occurring molecule, such as for example Fab, Fab′, and F(ab)₂ fragments of an antibody. Also contemplated for use in the methods of the invention are recombinant, truncated, single chain, chimeric, and hybrid antibodies, including, but not limited to, humanized and primatized antibodies, and other non-naturally occurring antibody forms.

A “ligand” is any molecule that binds to a specific site on another molecule, often a receptor.

The terms “patient,” “subject,” and “individual,” are used interchangeably herein, to refer to mammals, including, but not limited to, humans, murines, simians, felines, canines, equines, bovines, porcines, ovines, caprines, avians, mammalian farm and agricultural animals, mammalian sport animals, and mammalian pets.

“Biological sample,” as used herein, includes biological fluids such as blood, serum, plasma, urine, cerebrospinal fluid, tears, saliva, lymph, dialysis fluid, lavage fluid, semen, and other liquid samples or tissues of biological origin. It includes cells or cells derived therefrom and the progeny thereof, including cells in culture, cell supernatants, and cell lysates. It includes organ or tissue culture-derived fluids, tissue biopsy samples, tumor biopsy samples, stool samples, and fluids extracted from physiological tissues. Cells dissociated from solid tissues (e.g. tumors), tissue sections, and cell lysates are included. The definition also includes samples that have been manipulated in any way after their procurement, such as by treatment with reagents, solubilization, or enrichment for certain components, such as polynucleotides or polypeptides. Also included in the term are derivatives and fractions of biological samples. A biological sample can be used in a diagnostic or monitoring assay, and may be analyzed for BORIS expression products.

“Treatment,” as used herein, covers any administration or application of remedies for disease in an animal, including a human, and includes inhibiting the disease, i.e., arresting its development; relieving the disease, i.e., causing its regression; and eliminating the disease, i.e., causing the removal of diseased cells or restoration of a non-diseased state.

“Cancer” as used herein, refers to any abnormal cell or tissue growth, e.g., a tumor, which can be malignant or non-malignant. Cancer is characterized by uncontrolled proliferation of cells that may or may not invade the surrounding tissue and, hence, may or may not metastasize to new body sites. Cancer encompasses carcinomas, which are cancers of epithelial cells (e.g. squamous cell carcinoma, adenocarcinoma, melanomas, and hepatomas). Cancer also encompasses sarcomas, which are tumors of mesenchymal origin, (e.g. osteogenic sarcomas, leukemias, and lymphomas). Cancers can involve one or more neoplastic cell type.

A “pharmaceutical composition” or “pharmaceutically acceptable composition” of modulators, polypeptides, or polynucleotides herein refers to a composition that usually contains a pharmaceutically acceptable carrier or excipient that is conventional in the art and which is suitable for administration into a subject for therapeutic, diagnostic, or prophylactic purposes. For example, compositions for oral administration can form solutions, suspensions, tablets, pills, capsules, sustained release formulations, oral rinses, or powders.

The present invention is based on the observation that short interfering RNAs (siRNAs) are effective in inhibiting the expression of the Brother of the Regulator of Imprinted Sites (BORIS), which observations are detailed in commonly owned PCT International Application No. PCT/US08/72829, filed Aug. 11, 2008, the entire contents of which is incorporated by reference herein.

The present invention provides anti-tumor therapeutics capable of targeting and delivering an anti-tumor agent to a tumor stem cell by having a substantially higher affinity to a tumor stem cell than to other cells, particularly tumor non-stem cells. In one embodiment, compositions of the invention include a) a liposome, b) an antibody linked to the liposome, and c) an siRNA molecule capable of silencing BORIS.

In other embodiments of the invention, the anti-tumor therapeutics of the invention are targeted to tumor cells by having a substantially higher affinity to a tumor cell than to other cells, such as normal cells.

The generation of immunoliposomes is well-known in the art and described in numerous publications (see e.g., Zhang, et al. 2003, Pharm Res 20:1779-1785; Zhang, et al. 2002, Mol Ther 6:67-72; Zhang, et al. 2004, Clin Cancer Res 10:3667-3677; Zhang, et al. 2003, Mol Vis 9:465-472; Zhang, et al. 2003, J Gene Med 5:1039-1045; Shi, et al. 2001, Proc Natl Acad Sci USA 98:12754-12759; and Shi, et al. 2001, Pharm Res 18:1091-1095, the contents of which are incorporated herein by reference in their entirety for any purpose). The present invention provides compositions of immunoliposomes that target tumor stem cells, as well as methods for targeting tumor stem cells using the same. According to the present invention, tumor-targeting immunoliposomes are coated with antibodies specific to tumor stem cells. As used herein, a molecule, such as an antibody, that is “specific to tumor stem cells” means that such molecules has higher affinity to at least one tumor stem cell than it does to other cells that are not tumor stem cells. In one embodiment, the affinity of the molecule specific to tumor stem cells is at least about 2-10 fold higher than to other cells. In other embodiments, the affinity of a molecule specific to tumor stem cells is at least about 100, 1000, 10,000, or 100,000 fold higher than to other cells. In certain embodiments of the invention, the affinity of the molecule specific to tumor stem cells is at least about 2-10 fold higher than to tumor non-stem cells. In other embodiments, the affinity of a molecule specific to tumor stem cells is at least about 100, 1000, 10,000, or 100,000 fold higher than to tumor non-stem cells.

Exemplary antibodies suitable for use as the molecule that is specific to tumor stem cells in compositions of the present invention are directed to and bind antigens such as CD133, decay accelerating factor, CD117, prostate stem cell antigen, CD44, CD29, alpha6-integrin, CD200, stem cell antigen, and multiple drug resistance protein. According to one aspect of the present invention, at least one antibody specific to tumor stem cells is incorporated into an immunoliposome, via a procedure, such as a biochemical procedure, that is well-known in the art. In one embodiment, the procedure involves thiolation of the antibody to facilitate conjugation to immunoliposomes. The skilled artisan will be knowledgeable of other suitable procedures for incorporating antibodies into liposomes to prepare immunoliposomes.

Immunoliposomes containing antibodies targeted to or specific to tumor stem cells would be expected deliver a higher concentration of a therapeutic agent to a tumor stem cell than to other cells. However, such approaches have not been entirely satisfactory. Thus, in one embodiment the compositions of the present invention include an additional agent that selectively kills tumor stem cells but not non-malignant cells. For example, molecules capable of inhibiting an activity of BORIS can be inserted into the immunoliposomes specific to tumor stem cells. In certain aspects, the invention contemplates the use of RNA interference for treatment of cancer through the inhibition of expression of the BORIS transcription factor. In certain embodiments of the invention a patient with cancer is treated by administering short interfering RNA with a sequence homologous to the gene encoding BORIS, inserted into an immunoliposome specific to tumor stem cells.

siRNA at concentrations as low as the nanomolar range has been found effective in inhibiting BORIS gene expression. Accordingly, the present invention contemplates delivering siRNA to a tumor stem cell at an effective concentrations of 0.001 nM to greater than 50 μM; typically 0.01 nM to 5 μM; frequently 0.1 nM to 500 nM; and most often 1 nM to 50 nM.

The inhibition of expression of BORIS in the cell by the methods of the invention can result in at least about 10% inhibition (relative to the amount of BORIS in an untreated, control cell) within 1-5 days. In certain embodiments, at least about 20%, 40%, 60%, 80%, 90% or 95% inhibition of BORIS expression is obtained. In some embodiments of the invention, at least about 50-90% inhibition, at least about 60-95% inhibition, or at least about 70-99% inhibition of BORIS expression is observed. The percent inhibition of BORIS expression in a cell is typically determined by measuring the amount of BORIS polypeptide in a cell treated with a composition of the invention to the amount of BORIS polypeptide in an untreated control cell. Any method can be used to measure BORIS polypeptide, such as immunological methods e.g. western blotting, enzyme linked immunoassays (ELISAs), immunoprecipitation, immunofluorescence, FACS and other methods involving anti-BORIS antibodies or the like. BORIS gene expression may also be detected using biochemical techniques for analyzing RNA (e.g., mRNA) such as Northern blotting, nuclease protection assays, reverse transcription, and microarray hybridization, In other aspects of the invention, BORIS expression is determined by measuring an activity of BORIS, such as BORIS methylation activity, DNA binding activity, or cell transformation activity. In certain embodiments of the invention, the downstream effects of reduced BORIS gene expression may be measured as an indication of the inhibition of BORIS expression. Such downstream effects include reduced cell viability, cell death, increased apoptosis or the increased activity of apoptosis-related markers (e.g. caspases).

Any region of the BORIS nucleic acid sequence can be used as a target for designing the siRNAs of the invention, particularly regions of the mRNA sequence disclosed in GenBank under Accession Number AF336042 (deposited May 16, 2002). Preferably, the siRNA will be substantially identical to a BORIS nucleic acid sequence over a stretch of at least 10, 11, 12, 13, 14, 15, 16, 17, 18, 19, 20, 21, or 22 nucleotides. Typically, the target region is an exonic region that is towards the 5′ end of the targeted BORIS mRNA. A preferred target region of BORIS is the 11 zinc finger DNA binding region.

In certain embodiments, the siRNA is homologous to a 15-30 nucleotide target region of BORIS polynucleotide. Polynucleotide sequences suitable for siRNAs of the invention are set forth as SEQ ID NOs:1-61. In certain embodiments, sequences suitable for siRNAs of the invention are set forth as SEQ ID NOs:62-123. In one embodiment of the invention, the siRNA comprises OCM-8054 (SEQ ID NO:59). In another embodiment, the siRNA comprises OCM-8055 (SEQ ID NO:60).

In certain embodiments of the invention, the siRNAs are double-stranded RNAs, at least about 15-30 nucleotides in length, e.g., each complementary polynucleotide of the double-stranded siRNA is 15-30 nucleotides in length, and the double-stranded siRNA is about 15-30 base pairs in length, typically about 19-24 base nucleotides, most frequently about 21-22 nucleotides in length, that are prepared from chemically synthesized oligonucleotides and then introduced directly into the cell, e.g. by transfection. In some embodiments of the invention, the siRNA is a DNA-RNA chimera (having both ribo- and deoxyribonucleotides on a single oligonucleotide strand) or a DNA-RNA hybrid (in which one strand is DNA and the other is RNA).

The double-strand siRNAs of the invention may be blunt ended or have single nucleotide 5′ overhangs at one or both 5′ termini. However, it is known that the most potent silencing induced by administration of double-stranded RNA occurs when the duplexes have overhanging 3′ ends of 1-3 nucleotides. Thus, the siRNAs of the invention typically have overhangs at one or preferably both of its 3′ termini, these overhangs are preferably only a few nucleotides in length and in particular are one or two nucleotides in length, preferably two nucleotides in length. To provide an example, but not a limitation on the siRNA molecules of the invention, 21 nucleotide oligonucleotides that form a 19 nucleotide duplex region of base pairs with 2 nucleotide 3′-overhangs are very potent at stimulation of RNA interference. In certain embodiments, siRNAs of the invention have a 19 ribonucleotide duplex region with 2 deoxyribonucleotide 3′ overhangs on each end.

Chemically synthesized oligonucleotides suitable for use in the present invention can be prepared by any method known in the art. To increase the stability and/or improve the efficacy of the oligonucleotides in RNAi methods, modifications of the sugar, base or phosphodiester backbone can be incorporated. Non-limiting examples of such modifications include replacement of one or more phosphodiester bonds with phosphorothioate linkages; modifications at the 2′-position of the pentose sugar, such as incorporation of 2′-O-methyl ribonucleotides, 2′-H ribonucleotides, 2′-deoxy-2′-fluoro ribonucleotides (e.g. 2′-deoxy-2′-fluorouridine), or 2′-deoxy ribonucleotides; incorporation of universal base nucleotides, 5-C-methyl nucleotides, inverted deoxyabasic residues, or locked nucleic acid (LNA), which contains a methylene linkage between the 2′ and the 4′ position of the ribose. Additional chemical modifications of siRNA molecules contemplated for use in the present invention are described in Corey (J. Clin. Invest. (2007) 12:3615-22), the contents of which are incorporated by reference herein); other suitable modifications will be well known to the skilled artisan. Such chemical modifications, when incorporated into the strands of double-stranded RNA, have been shown to potentiate or preserve the ability to induce RNA interference in the target cells while at the same time, dramatically increasing the serum stability of the molecules.

Alternatively, template polynucleotides can be prepared that encode the “sense” and “anti-sense” strand of the siRNA molecules of the invention. In certain aspects, the template polynucleotides of the invention are used to enzymatically synthesize the complementary strands of the siRNA in vitro. In other aspects, the polynucleotide can be, for example, transfected into a cell for intracellular synthesis of the siRNA. In these aspects of the invention, introduction of siRNA into the cell is indirect in that a template polynucleotide is introduced into a cell, which then serves as a template for synthesis of the siRNA strands using cellular machinery, but has the effect of introducing siRNA into the cell.

Accordingly, the present invention provides template polynucleotides for directing synthesis of interfering RNAs both in vitro and in vivo. In certain embodiments of the invention, the template polynucleotides encode the complementary strands of siRNAs, which are not greater than 30 nucleotides in length, are typically are 19-24, and frequently 21-22 nucleotides in length. Such template polynucleotides are particularly useful for in vitro synthesis of siRNA oligonucleotides. The skilled artisan will be knowledgeable in recombinant DNA methods for constructing polynucleotides that can be transcribed in vitro to produce the desired oligonucleotide products.

In other embodiments, the polynucleotides of the invention encode siRNA precursors, such as the complementary strands of longer double-strand interfering RNA molecules, or short hairpin RNAs (shRNAs), which mimic naturally occurring precursor microRNAs (miRNAs) and are efficiently processed by the mammalian cellular machinery into active siRNA. While not wishing to be bound by a particular theory, miRNAs are believed to be endogenous substrates for the RNAi machinery, which are initially expressed as long primary transcripts (pri-miRNAs), and then processed into 60-70 bp hairpins. Finally, the loop of the hairpin is removed resulting in siRNAs.

Thus, the present invention also provides polynucleotide templates for shRNA as well as templates for one or both strands of an siRNA. The shRNA templates typically include a promoter directly followed by at least about 18 nucleotides, typically 19, 20, 21 or 22 nucleotides, of sense (or antisense) target sequence, a 4-13 nucleotide loop, the complementary antisense (or sense) target sequence and finally a stretch of at least four to six U's as a terminator. The sense and anti-sense sequences are complementary but may not be completely symmetrical, as the hairpin structure may contain 3′ or 5′ overhang nucleotides (e.g., 1, 2, 3, 4, or 5 nucleotide overhangs). Similar templates for siRNAs can be produced, for example, by placing sense and antisense target sequences under the control of their own promoters in the same construct, without an intervening loop.

The promoter will be operably linked to the region encoding the siRNA, shRNA or other interfering RNA. Typically, the RNA coding sequences will be immediately downstream of the transcriptional start site or be separated by a minimal distance such as less than about 20 base pairs, typically less than about 10 base pairs, frequently less than about 5 base pairs and most often 2 two or fewer base pairs. “Operably linked,” as used herein, means without limitation, that the RNA coding region is in the correct location and orientation with respect to the promoter such that expression of the gene will be effected when the promoter is contacted with the appropriate polymerase and any required transcription factors.

The promoter may be any suitable promoter for directing transcription of the shRNA or siRNA. In certain embodiments, the promoter is an RNA polymerase III (pol III) promoter. A suitable range of RNA polymerase III promoters are described, for example, in Paule & White (Nucleic Acids Res. (2000) 28:1283-98), which is incorporated by reference herein in its entirety. RNA polymerase III promoters include any naturally occurring, synthetic or engineered DNA sequence that can direct RNA polymerase III to transcribe downstream RNA coding sequences. The RNA polymerase III promoter or promoters used in the constructs of the invention can be inducible. Particularly suitable pol III promoters include those from H1 RNA, 5S, U6, adenovirus VA1, Vault, telomerase RNA, and tRNA genes, as well as the tetracycline responsive promoters described in Ohkawa & Taira (Human Gene Ther. (2001) 11:577-85) and in Meissner et al. (Nucleic Acids Res., (2001) 29:1672-82), which are incorporated herein by reference.

In other embodiments, the promoter is recognized by RNA polymerase II (pol II). A wide variety of pol II promoters are known in the art, including many cell-specific and inducible promoters. Use of such cell-specific and inducible promoters may be desirable as a mechanism for limiting RNAi effects to a particular cell type or controlling the timing of expression.

The template polynucleotides of the invention can be cloned into vectors, including but not limited to plasmid, cosmid, phagemid, and viral vectors according to well-known methods. The vectors can then be introduced into target cells that express BORIS where e.g, the siRNA produced therefrom directs cleavage of BORIS mRNA and thereby inhibits BORIS expression. The skilled artisan will appreciate that bacterial, bacteriophage, insect, fungal and other non-mammalian vectors may provide suitable templates for introduction into cultured mammalian cells. For clinical applications, a vector capable of persistence in the target cell, such as a viral vector, may be more desirable. Viral vectors also offer the advantage of efficient transfer of the template polynucleotide into the cell via infection rather than transfection. Exemplary viral vectors for clinical applications of the invention include but are not limited to adenoviruses, adeno-associated viruses, retroviruses, lentiviruses, vaccinia viruses, herpes viruses, and papilloma viruses.

In yet another embodiment, siRNAs suitable for use in the invention can be prepared by enzymatic digestion of a longer double-strand RNA using an RNase III type enzyme (e.g., Dicer). Commercially available Dicer siRNA generation kits are currently available, permitting synthesis of large numbers of siRNAs from full length target genes (Gene Therapy Systems, Inc, MV062603).

The present invention also provides combination therapy for cancer. Thus, the compositions of the invention can be coupled with traditional surgical removal of tumor tissue, radiation therapy, immunotherapeutic treatment and/or chemotherapeutic methods for treating cancer. In one embodiment, surgical removal of a tumor is accompanied by localized instillation of the surrounding area with siRNA of the invention.

In another embodiment, an immunotherapeutic agent, such as an innate immune stimulator, a stimulator of adaptive immunity, or both an innate immune stimulator and a stimulator of adaptive immunity, is co-administered with the compositions of the invention, which can be e.g. simultaneous or sequential co-administration. The innate immune stimulator can, for example, activate immune functions through the upregulation of biological function of cells such as dendritic cells, macrophages, neutrophils, mast cells, natural killer cells, natural killer T cells, gamma delta cells, and B1 B cells. The stimulator of adaptive immunity can be, for example a peptide vaccine, a protein vaccine, an altered peptide-ligand vaccine, a DNA vaccine, an RNA vaccine, a cell therapy, or a dendritic cell vaccine. In certain aspects, the vaccine stimulates a T cell response against an epitope of the BORIS protein.

In another embodiment, the compositions of the invention are administered in combination (e.g. sequential or simultaneous administration in a pharmaceutically acceptable composition) with at least one typical therapeutic or palliative anticancer drug, which include, without limitation alkylating agents such as thiotepa, and cyclosphosphamide; alkyl sulfonates such as, busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunomomycin, daunorubicin, detorubicin, 6-diazo-5-oxo-L-norleucine, doxorubicin, epirubicin, esorubicin, idarubicin, marcellomycin, mitomycins (e.g., mitomycin C), mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); taxanes, paclitaxel and docetaxel; gemcitabine; platinum analogs such as cisplatin and carboplatin; etoposide; mitoxantrone; anti-mitotics; vinblastine; vincristine; vinorelbine; novantrone; teniposide; aminopterin; ibandronate; iretotecan; topoisomerase inhibitor RFS 2000; difluoromethylomithine (DMFO); retinoic acid; esperamicins; capecitabine; abarelix; aldesleukin; aldesleukin; alemtuzumab; alitretinoin; allopurinol; amifostine; anakinra; anastrozole; arsenic trioxide; asparaginase; bcg live; bevacizumab; bexarotene; bleomycin; bortezomib; celecoxib; cetuximab; cladribine; clofarabine; dalteparin sodium; darbepoetin alfa; dasatinib; daunomycin; decitabine; denileukin; dexrazoxane; eculizumab; elliott's b solution; epoetin alfa; erlotinib; exemestane; fentanyl citrate; filgrastim; fulvestrant; gefitinib; gemtuzumab ozogamicin; goserelin acetate; histrelin acetate; ibritumomab tiuxetan; imatinib mesylate; interferon alfa 2a; irinotecan; lapatinib; ditosylate; lenalidomide; letrozole; leucovorin; leuprolide; acetate; levamisole; ccnu; meclorethamine; megestrol; acetatemesna; methoxsalen; nandrolone phenpropionate; nelarabine; nofetumomab; oprelvekin; oxaliplatin; palifermin; pamidronate; panitumumab; pegademase; pegaspargase; pegfilgrastim; peginterferon alfa-2b; pemetrexed disodium; plicamycin; mithramycin; porfimer sodium; quinacrine; rasburicase; rituximab; sargramostim; sorafenib; sunitinib; tamoxifen; thalidomide; topotecan; topotecan hcl; toremifene; tositumomab; trastuzumab; tretinoin; atra; valrubicin; vorinostat; zoledronate; zoledronic acid; decitabine; aprepitant; imiquimod; ixabepilone; letrozole; oxaliplatin; raloxifene; rituximab; sorafenib tosylate; tarabine pfs; erlotinib; nilotinib; docetaxel; temozolomide; temsirolimus; bendamustine hydrochloride; lapatinib ditosylate; leuprolide acetate; and dexrazoxane hydrochloride.

The invention will now be further exemplified by the following non-limiting examples, including the experiments conducted and results achieved, which are provided for illustrative purposes only and are not to be construed as limiting the present invention in any way.

### EXAMPLE[S]

### Example 1

### Preparation of CD133 Targeted siRNA-Bearing Immunoliposomes

1-Palmitoyl-2-oleoyl-sn-glycerol-3-phosphocholine (POPC), dimethyldioctadecylammoniumbromide (DDAB), distearoylphosphatidylethanolamine-PEG²⁰⁰⁰ (DSPE-PEG²⁰⁰⁰), and PEG²⁰⁰⁰ Dalton polyethyleneglycol distearoylphosphatidylethanolamine-PEG²⁰⁰⁰-maleimide (DSPE-PEG²⁰⁰⁰-Mal) are dissolved in choloroform and mixed at molar ratios of 92:4:3:1 (for neutral liposomes), 91:5:3:1 (for 1 mole % positive liposomes), or 90:6:3:1 (for 2 mole % positive liposomes), respectively. The total amount of lipid used is 20.2 μmol. The chloroform-dissolved lipids are mixed together in a conical glass flask and the chloroform is evaporated using a sterile nitrogen gas stream, leaving a thin lipid film coating the walls of the flask. Lipids are then placed in a vacuum centrifuge for 90 min to remove residual chloroform. 250 μg of BORIS-targeting siRNA is dissolved in 0.05 M Tris-HCL (pH 8.0) to a final volume of 0.2 ml, which is subsequently added to the lipid film. The mixture is subsequently vortexed for 5 min and sonicated for 2 min using a bath sonicator. Subsequently, the mixture is frozen by submersion in liquid nitrogen and thawed at room temperature. The freeze/thaw cycle is repeated 6 times. Liposomes are diluted to a concentration of 40 mM by adding 0.05 M HEPES, pH 7.0 (0.3 ml) and subsequently passed through 2 stacked polycarbonate membranes of 400 nm pore size. This is repeated using 200 nm, 100 nm, and 50 nm pore size polycarbonate membranes. siRNA molecules on the outside of the liposome are degraded using RNase III. Specifically, ShortCut RNase buffer (10% v/v), MnCl₂ (10% v/v), and 20 units of ShortCut RNase III is used to treat the liposome/RNA dispersion. The digestion reaction mixture is incubated at 37° C. for 2 hours, and then the reaction is stopped by adding 20 mM EDTA (10% v/v). The immunoliposome mixture is passed through a 1.5×10 cm Sepharose CL-4B column to separate siRNA-bearing immunoliposomes from digested siRNA fragments and un-conjugated antibody. Fractions (approximately 26-30) of 0.5 ml each are collected and the fluorescence (excitation/emission 550/570 and 650/668) of each eluted fraction is determined using a mass spectrofluorometer. Fractions corresponding to the first set of overlapping fluorescence peaks which exhibit co-fractionation of antibody and siRNA, are pooled and concentrated using a Centricon filtration device with a 100 KDa MWCO. siRNA fluorescence is measured once again and compared against a standard curve to determine the final concentration of encapsulated siRNA. The preparation is then filter sterilized using a 0.2 μm filter (Millipore, Billerica, Mass.). 3 mg of anti-CD133 antibody is dissolved in 0.15 M Na-borate/0.1 mM EDTA (pH 8.5) and thiolated for 1 hour at room temperature using 2-iminothiolane (Traut's Reagent) at a 40:1 molar excess ratio. The buffer is then exchanged with 0.05 M HEPES/0.1 mM EDTA (pH 7.0) using a Centricon YM-30 ultracentrifugal filtration device and the antibody is immediately used for conjugation to liposomes. Thiolated antibody is added to the liposome dispersion and the mixture incubated overnight at room temperature.

### Example 2

### Specificity for Tumor Stem Cell

Surgical samples are obtained from patients with stage 1V, poorly differentiated colon cancer. Cells are mechanically dissociated and incubated with Collagenase Type IV to prepare a single cell suspension. Cells are washed in phosphate buffered saline followed by magnetic bead separation to purify cells expressing CD133 using a magnetic activated cell sorting (MACS) system. In order to extract tumor cells lacking CD133, the unselected cells are also harvested. Cells are cultured in DMEM media supplemented with 10% fetal calf serum and penicillin/streptomycin in 96 well plates. Subsequent to overnight plating, non-adherent cells are washed off with PBS, and immunoliposomes are administered. Control immunoliposomes are generated with a thiolated IgG control antibody, whereas immunoliposomes specific to tumor stem cells are generated with thiolated anti-CD133 antibody. One group of control and tumor specific immunoliposes are loaded with siRNA sequence targeting BORIS (SEQ ID NO:60), whereas another group are loaded with control scrambled siRNA. CD133 positive and CD133 negative cells are treated with 3 escalating doses of immunoliposomes. Per well, concentrations of immunoliposomes added are 10, 50 and 100 nanograms. Cells are incubated at 37° C. in a humidified, 5% carbon dioxide environment. At 48 hours, apoptosis is assessed by flow cytometric detection of Annexin-V-FITC conjugate. CD133 positive cells undergo a dose dependent increase in apoptosis in comparison to CD133 negative cancer cells. Treatment with immunoliposomes loaded with control scrambled siRNA does not lead to apoptosis.

### Example 2

### In Vivo Anti-Tumor Effect of Immunoliposomes Targeting CD133 Loaded with BORIS-Specific siRNA

Immunoliposomes are prepared as described in Example 1. A human-SCID model of colon cancer is prepared as described in O'Brien, et al. (2007, Nature 445:106-110). Briefly, primary patient samples are extracted from stage 1V colon cancer patients. Samples used are from patients with poorly to moderately differentiated tumors. Tumor tissue is degraded using collagenase IV and mechanically dissociated in order to obtain single cell suspensions. Viability of the single cell suspensions is assessed and a population of 10 million CD133-purified cells are administered underneath the kidney capsule. Tumors are allowed to grow for a period of 4 weeks. Recipient mice are severe combined immunodeficient (SCID) backcrossed into the non-obese diabetic strain. Subsequent to the 4 week period of engraftment, 10 mice are treated with an intravenous bolus of BORIS-specific siRNA loaded in CD133 immunoliposomes, 10 mice are treated with scrambled control siRNA loaded in CD133 bearing immunoliposomes, 10 mice are treated with BORIS-specific siRNA immunoliposomes bearing an isotype control IgG antibody, and 10 mice are treated with empty immunoliposomes coated with CD133. After an additional 4 weeks all mice are sacrificed. Tumors are substantially reduced only in mice that received the anti-CD133 coated BORIS siRNA-specific immunoliposome.

What is claimed is:

1. A composition for the treatment of cancer comprising:
a) at least one molecule specific to a tumor stem cell;
b) a carrier bound to the at least one molecule of a); and
c) at least one molecule capable of suppressing transcription, translation or a function of i) the Brother of the Regulator of Imprinted Sites (BORIS) molecule, or ii) an isoform of BORIS.

2. The composition of claim 1, wherein a) is an antibody, an aptamer, a fusion protein, or a small organic compound.

3. The composition of claim 2, wherein the antibody recognizes CD133, decay accelerating factor, CD117, prostate stem cell antigen, CD44, CD29, alpha6-integrin, CD200, stem cell antigen, or multiple drug resistance protein.

4. The composition of claim 1, wherein b) comprises at least one of: a liposome, a fullerene molecule, a cationic lipid particle, a biodegradable nanoparticle, or an aerosolized particle.

5. The composition of claim 1, wherein c) is an antisense oligonucleotide, a short interfering RNA, a ribozyme, a molecule that prevents BORIS from binding to DNA, a molecule that prevents binding of BORIS to co-factors, a molecule that prevents recruitment of cofactors needed for BORIS transcription.

6. The composition of claim 5, wherein the c) is a peptide or a small organic compound.

7. The composition of claim 1, comprising a polyethelyne glycol-based immunoliposome containing at least one anti-CD133 antibody and loaded with siRNA targeting the BORIS gene.

8. The composition of claim 7, wherein the antibody is thiolated to facilitate conjugation to the immunoliposome.

9. The composition of claim 7, wherein one strand of the siRNA has a nucleotide sequence selected from SEQ ID NOs:1-61.

10. The composition of claim 7, wherein one strand of the siRNA has a nucleotide sequence selected from SEQ ID NOs:62-123.

11. The composition of claim 9, wherein the siRNA molecule is synthesized from a polynucleotide that encodes the siRNA molecule or a precursor of the siRNA.

12. The composition of claim 1, wherein b) further comprises at least one molecule specific to a tumor cell.

13. A method of treating cancer comprising administering the composition of claim 1 to a subject.

14. A method of treating cancer comprising administering the composition of claim 12 to a subject.

15. The method of claim 13, further comprising administering to the subject at least one of: a chemotherapeutic agent, an immunotherapeutic agent, a hormonal therapeutic agent, radiation therapy, surgery, and embolization therapy.

16. The method of claim 14, further comprising administering to the subject at least one of: a chemotherapeutic agent, an immunotherapeutic agent, a hormonal therapeutic agent, radiation therapy, surgery, and embolization therapy.

17. A composition for the treatment of cancer consisting of:
a) an immunoliposome comprising a thiolated antibody that binds CD133 coupled to the distal reactive maleimide terminus of a poly(ethylene glycol)-phospholipid conjugate so that the antibody is partially incorporated into liposomal bilayer; and
b) a nucleic acid sequence capable of selectively inhibiting expression or an activity of BORIS, wherein b) is encapsulated by a).

18. The composition of claim 17, wherein b) has the nucleotide sequence of SEQ ID NOs:59 or 60.

19. An admixture comprising the composition of claim 17 admixed with a cytotoxic agent.

20. The composition of claim 17, wherein the immunoliposome has a particle size of about 50 to a about 400 nanometers.
